(19) United States
(12) Patent Application Publication
(10) Pub. No.: US 2026/0240589 A1
(43) Pub. Date: Aug. 20, 2026

(54) SURGICAL END EFFECTOR ASSEMBLIES AND SURGICAL INSTRUMENTS FOR ENERGY-BASED TISSUE CUTTING

(71) Applicant: Covidien LP, Mansfield, MA (US)

(72) Inventors: Dylan R. Kingsley, Broomfield, CO (US); Daniel W. Mercier, Erie, CO (US); Jake H. Young, Superior, CO (US); Craig V. Krastins, Arvada, CO (US); Chelsea E. Walbridge, Longmont, CO (US); David J. Van Tol, Boulder, CO (US)

(21) Appl. No.: 19/489,494

(22) PCT Filed: May 30, 2024
(86) PCT No.: PCT/IB2024/055284

§ 371 Date: Dec. 2, 2025

Related U.S. Application Data
(60) Provisional application No. 63/471,076, filed on Jun. 5, 2023.

(51) Int. Cl. A61B 18/14; A61B 18/00; A61B 34/30

(52) U.S. Cl. CPC A61B 18/1445; A61B 34/30; A61B 2018/00601; A61B 2018/1452

(57) ABSTRACT

A surgical end effector assembly includes first and second jaw members configured to grasp tissue therebetween. The second jaw member includes a cutting electrode. The first jaw member includes a jaw body, a compression pad, and at least one spring operably coupling the compression pad with the jaw body. The first and second jaw members are configured to cooperate to grasp tissue between the cutting electrode and the compression pad.

### CROSS-REFERENCE TO RELATED APPLICATION

This application claims the benefit of U.S. Provisional Patent Application Ser. No. 63/471,076, filed Jun. 5, 2023, the entire content of which is incorporated herein by reference.

### FIELD

This disclosure relates to surgical instruments and, more specifically, to surgical end effector assemblies and surgical instruments for energy-based tissue cutting such as, for example, for use in surgical robotic systems.

### BACKGROUND

Surgical robotic systems are increasingly utilized in various different surgical procedures. Some surgical robotic systems include a console supporting a robotic arm. One or more different surgical instruments may be configured for use with the surgical robotic system and selectively mountable to the robotic arm. The robotic arm provides one or more inputs to the mounted surgical instrument to enable operation of the mounted surgical instrument.

A surgical forceps, one type of instrument capable of being utilized with a robotic surgical system, relies on mechanical action between its jaw members to grasp, clamp, and constrict tissue. Electrosurgical forceps utilize both controlled mechanical clamping action and energy to heat tissue to seal (or otherwise treat) tissue. Typically, once tissue is sealed, the tissue is severed using a cutting element. Accordingly, many electrosurgical forceps are designed to incorporate a mechanical cutting element to effectively sever sealed tissue (and/or to cut tissue independently of tissue sealing). Alternatively, surgical forceps may incorporate an energy-based, e.g., thermal, electrical, ultrasonic, etc., cutting mechanism to cut tissue, whether previously sealed or unsealed.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of this disclosure is a surgical end effector assembly including first and second jaw members. The first and second jaw members include respective first and second tissue contacting surfaces. At least one of the first or second jaw members is movable relative to the other of the first or second jaw members between a spaced apart position and an approximated position. The second jaw member includes a cutting electrode extending from the second jaw member towards the first jaw member. The first jaw member includes a jaw body defining a slot, a compression pad at least partially disposed within the slot, and at least one spring operably coupling the compression pad with the jaw body.

In aspects of this disclosure, the at least one spring extends longitudinally along the first jaw member. In aspects of this disclosure, the at least one spring extends transversely across the first jaw member. In aspects of this disclosure, the at least one spring operably couples the compression pad with the jaw body to define a varied effective durometer of the compression pad in at least one dimension thereof.

In an aspect of this disclosure, the at least one spring includes a cantilever spring. The cantilever spring may include a fixed proximal end portion fixed to the jaw body and a free distal end portion that extends distally into the slot. Alternatively, the cantilever spring may include a fixed distal end portion fixed to the jaw body and a free proximal end portion that extends proximally into the slot. The cantilever spring may extend longitudinally along the first jaw member or transversely across the first jaw member. The cantilever spring may extend partially into the compression pad and terminate at a free end portion within the compression pad. The cantilever spring may operably couple the compression pad with the jaw body to define a varied effective durometer of the compression pad in at least one dimension thereof.

In an aspect of this disclosure, the at least one spring includes an arched spring. The arched spring may define a convex configuration oriented towards the second jaw member. The arched spring may extend longitudinally along the first jaw member. Alternatively, the arched spring may extend transversely across the first jaw member. The arched spring may define first and second end portions and an arched body portion disposed therebetween. The arched body portion may extend through the compression pad. The first and second end portions of the arched spring may be fixed to the jaw body. The arched spring may operably couple the compression pad with the jaw body to define a varied effective durometer of the compression pad in at least one dimension thereof.

In an aspect of this disclosure, the at least one spring includes a first spring coupled to the jaw body and a second spring coupled to the compression pad. In such aspects, the first and second springs may operably couple the compression pad with the jaw body. The first and second springs may be oppositely oriented relative to one another. The first and second springs may be arched springs coupled to one another at apexes thereof. The first and second springs may extend longitudinally along the first jaw member. Alternatively, the first and second springs may extend transversely across the first jaw member. The first and second springs may operably couple the compression pad with the jaw body to define a varied effective durometer of the compression pad in at least one dimension thereof.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with the present disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIGS. 8A and 8B are side views of a portion of the end effector assembly of the surgical instrument of FIG. 5 with jaw members of the end effector assembly disposed in spaced apart and approximated positions, respectively;

FIG. 9 is a transverse, cross-sectional view of the end effector assembly of the surgical instrument of FIG. 5 with the jaw members of the end effector assembly disposed in the approximated position;

FIG. 10A is a longitudinal, cross-sectional view of a distal portion of one of the jaw members of the end effector assembly of the surgical instrument of FIG. 5 including a compression pad operably engaged within the jaw member in accordance with aspects of this disclosure;

FIG. 10B is a longitudinal, cross-sectional view of a distal portion of one of the jaw members of the end effector assembly of the surgical instrument of FIG. 5 including a compression pad operably engaged within the jaw member in accordance with other aspects of this disclosure;

FIG. 11 is a longitudinal, cross-sectional view of a distal portion of one of the jaw members of the end effector assembly of the surgical instrument of FIG. 5 including a compression pad operably engaged within the jaw member in accordance with still other aspects of this disclosure;

FIG. 12 is a longitudinal, cross-sectional view of a distal portion of one of the jaw members of the end effector assembly of the surgical instrument of FIG. 5 including a compression pad operably engaged within the jaw member in accordance with yet other aspects of this disclosure;

FIG. 13 is a transverse, cross-sectional view of a distal portion of one of the jaw members of the end effector assembly of the surgical instrument of FIG. 5 including a compression pad operably engaged within the jaw member in accordance with still yet other aspects of this disclosure;

FIG. 14 is a transverse, cross-sectional view of a distal portion of one of the jaw members of the end effector assembly of the surgical instrument of FIG. 5 including a compression pad operably engaged within the jaw member in accordance with aspects of this disclosure; and FIG. 15 is a transverse, cross-sectional view of a distal portion of one of the jaw members of the end effector assembly of the surgical instrument of FIG. 5 including a compression pad operably engaged within the jaw member in accordance with other aspects of this disclosure.

### DETAILED DESCRIPTION

This disclosure provides surgical end effector assemblies and surgical instruments for energy-based tissue cutting. As described in detail below, the surgical end effector assemblies and surgical instruments of this disclosure are configured for use with a surgical robotic system, which may include, for example, a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which, in turn, move the robotic arm in response to the movement command. Those skilled in the art will understand that this disclosure, although described in connection with surgical robotic systems, may also be adapted for use with handheld surgical instruments such as, for example, endoscopic surgical instruments and/or open surgical instruments, whether manually operated or powered.

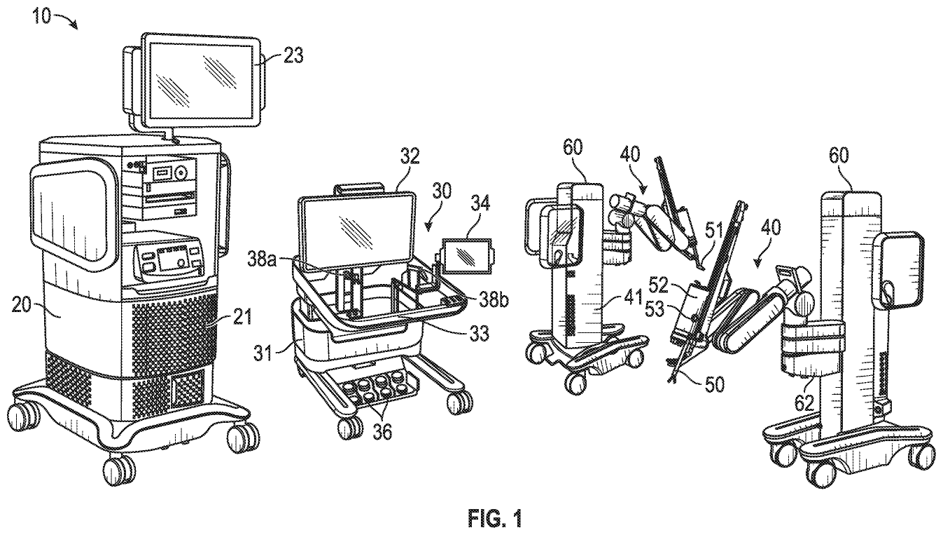

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50, 51 removably coupled thereto. Each of the robotic arms 40 is also coupled to a movable cart 60.

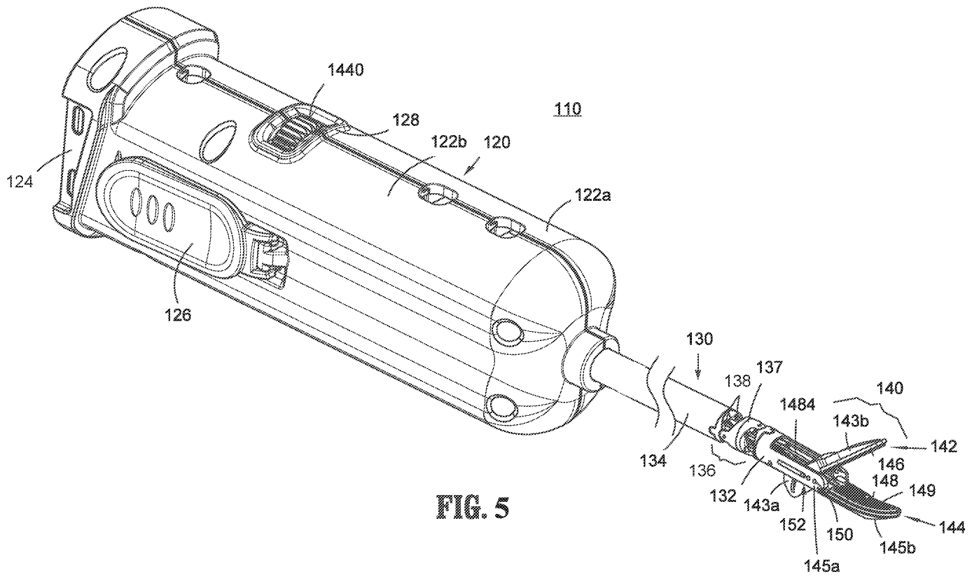

The one or more surgical instruments 50, 51 may be configured for use during minimally invasive surgical procedures and/or open surgical procedures. In aspects, one of the surgical instruments 50 may be an endoscope, such as an endoscope camera 51, configured to provide a video feed for the clinician. In aspects, one of the surgical instruments 50 may be an energy based surgical instrument such as, for example, an energy-based forceps configured to seal tissue by grasping tissue between opposing structures and applying energy, e.g., electrical, thermal, ultrasonic, light, etc., energy thereto and to cut tissue by applying energy, e.g., electrical, thermal, ultrasonic, light, etc., energy thereto. An example of such an energy-based forceps for energy-based sealing and cutting is described in detail below and identified by reference numeral 110 (FIG. 5).

Endoscope camera 51 is configured to capture video of the surgical site. The surgical console 30 includes a first display 32, which displays a video feed of the surgical site provided by endoscope camera 51, and a second display 34, which displays a user interface for controlling the surgical robotic system 10. The first and second displays 32 and 34 are touchscreens allowing for display of and interaction with various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, 51 based on a set of programmable instructions and/or input commands from the surgical console 30, in such a way that robotic arms 40 and the surgical instruments 50, 51 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth ® (an open wireless protocol for exchanging data over short distances, using short length radio waves, from fixed and mobile devices, creating personal area networks (PANs)), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

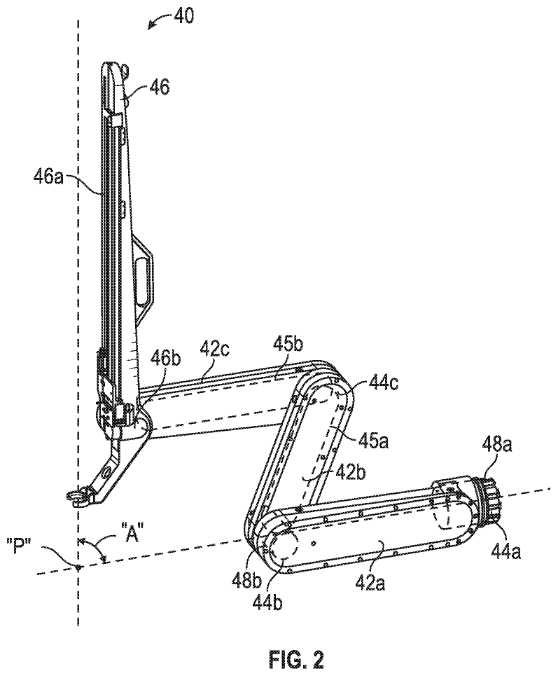

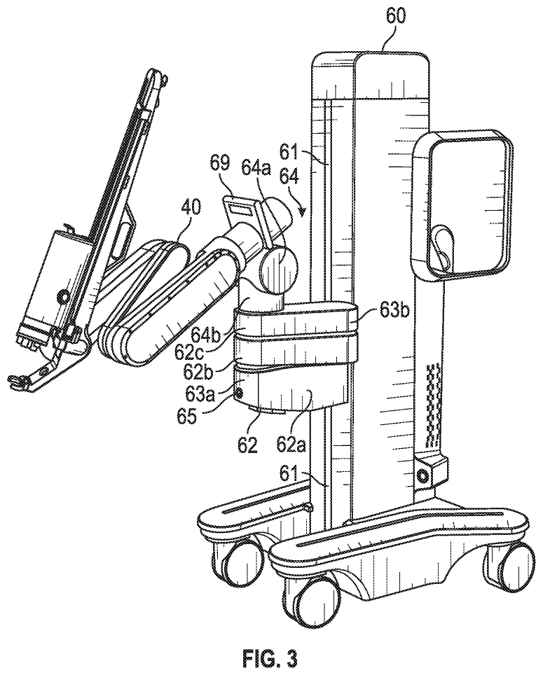

With reference to FIGS. 2 and 3, each of the robotic arms 40 may include a plurality of links 42a, 42b, 42c, which are interconnected at joints 44a, 44b, 44c, respectively. Joint 44a is configured to secure the robotic arm 40 to the movable cart 60 and defines a first longitudinal axis. The movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62a and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In aspects, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIGS. 1 and 2, the robotic arm 40 also includes a holder 46 defining a second longitudinal axis and configured to receive an IDU 52. The IDU 52 is configured to couple to an actuation mechanism of the surgical instrument 50 and the endoscope camera 51 and is configured to move (e.g., rotate) and actuate the instrument 50 and/or the endoscope camera 51. IDU 52 transfers actuation forces from its actuators to the surgical instrument 50 and/or the endoscope camera 51to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 further includes a plurality of manual override buttons 53 disposed on the IDU 52 and/or the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a remote center point “P” which lies at an intersection of the first axis defined by the link 42a and the second axis defined by the holder 46. Thus, the actuator 48b controls the angle “A” between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle “A.” In aspects, some or all of the joints 44a, 44b, 44c may include an actuator to obviate the need for mechanical linkages.

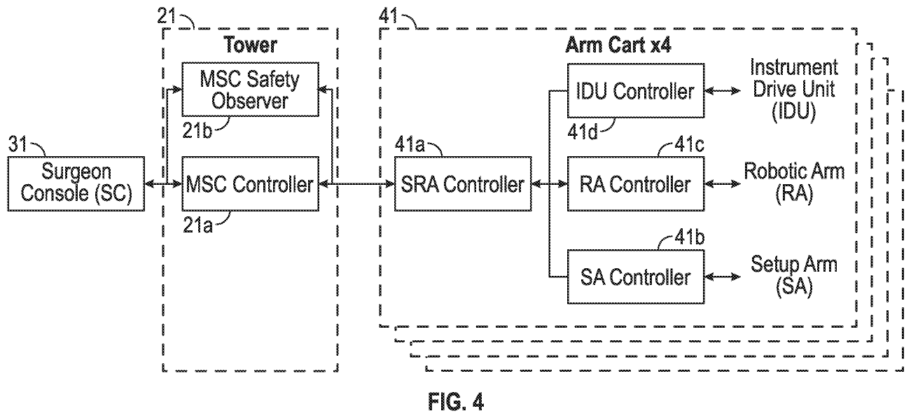

With reference to FIGS. 1 and 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The handle controllers 38a and 38b include one or more haptic feedback vibratory devices that output haptic feedback. The safety observer 21b performs validity checks on the data going into and out of the controller 2la and notifies a system fault handler if errors in the data transmission are detected to place the computer 21 and/or the surgical robotic system 10 into a safe state.

The computer 41 includes a plurality of controllers, namely, a main cart controller 4la, a setup arm controller 41b, a robotic arm controller 41c, and an IDU controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

With additional reference to FIGS. 2 and 3, the setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In aspects, the coordinate position is scaled down and the orientation is scaled up by the scaling function. In addition, controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

Turning to FIGS. 5-7, a surgical instrument 110 provided in accordance with the present disclosure generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with the present disclosure, detailed below, are equally applicable for use with other suitable surgical instruments, e.g., graspers, staplers, clip appliers, and/or in other suitable surgical systems, e.g., motorized, other power driven systems, and/or manually actuated surgical systems (including handheld instruments).

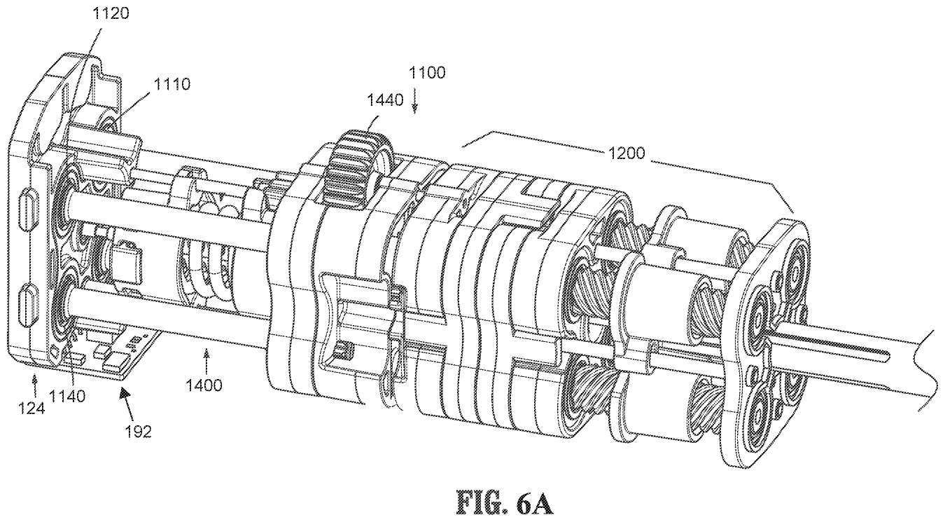

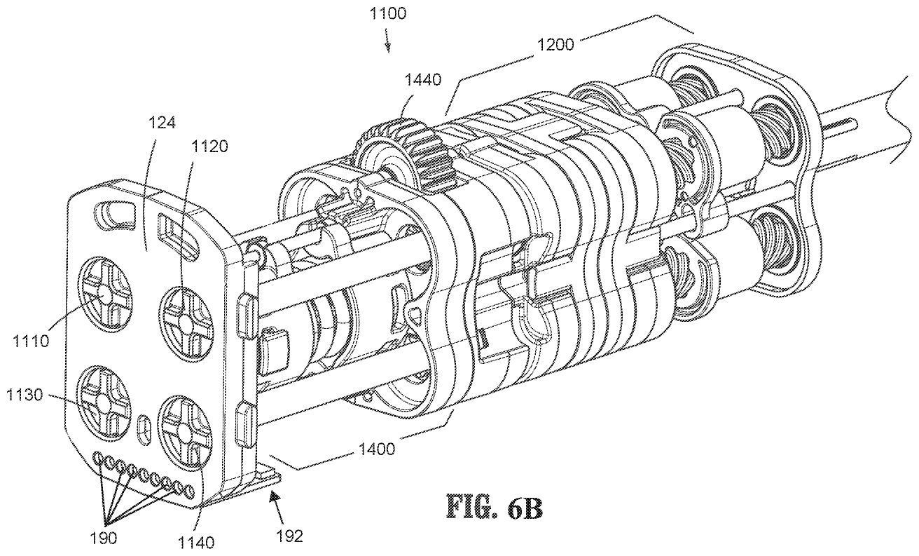

With particular reference to FIG. 5, housing 120 of instrument 110 includes first and second housing parts 122a, 122b and a proximal face plate 124 that cooperate to enclose actuation assembly 1100 therein. Proximal face plate 124 includes through holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm 40 (FIG. 1) of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

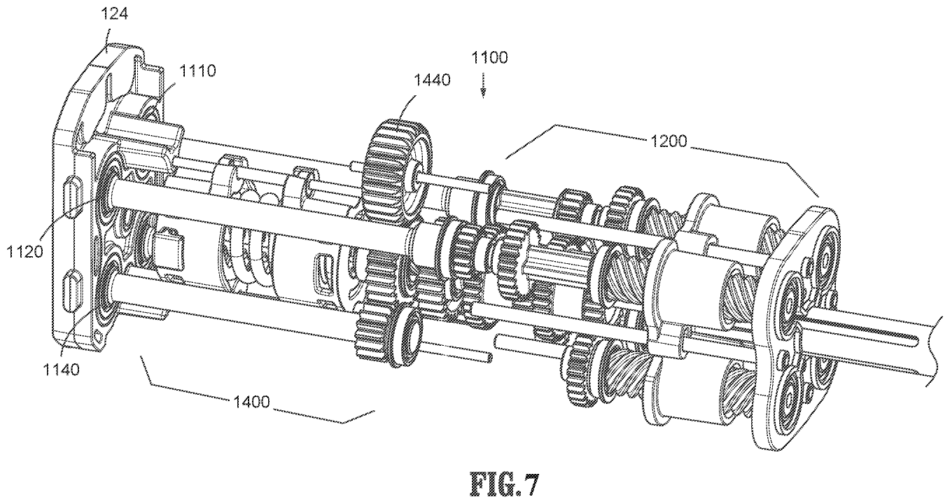

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer provided updates manually or automatically upon connection to the surgical robotic system 10 (FIG. 1).

Referring again to FIG. 5, shaft 130 of instrument 110 includes a distal clevis segment 132, a proximal segment 134, and an articulating section 136 disposed between the distal clevis and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal clevis segment 132 of shaft 130 at the distal ends thereof and extend proximally from distal clevis segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal clevis segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a proximal flange 143a, 145a and a distal body 143b, 145b, respectively. Distal bodies 143b, 145b define opposed tissue contacting surfaces 146, 148, respectively. Proximal flanges 143a, 145a are pivotably coupled to one another about a pivot 150 and are operably coupled to one another via a cam slot assembly 152 including a cam pin slidably received within cam slots defined within the proximal flange 143a, 145a of at least one of the jaw members 142, 144, respectively, to enable pivoting of jaw member 142 relative to jaw member 144 and distal segment 132 of shaft 130 between a spaced apart position (e.g., an open position of end effector assembly 140) and an approximated position (e.g., a closed position of end effector assembly 140) for grasping tissue between tissue contacting surfaces 146, 148. As an alternative to this unilateral configuration, a bilateral configuration may be provided whereby both jaw members 142, 144 are pivotable relative to one another and distal segment 132 of shaft 130. Alternatively, the above detailed configuration may be reversed, e.g., wherein jaw member 142 is the fixed jaw member and jaw member 144 is movable relative to jaw member 142. Other suitable jaw actuation mechanisms (for bilateral and/or unilateral jaw configurations) are also contemplated.

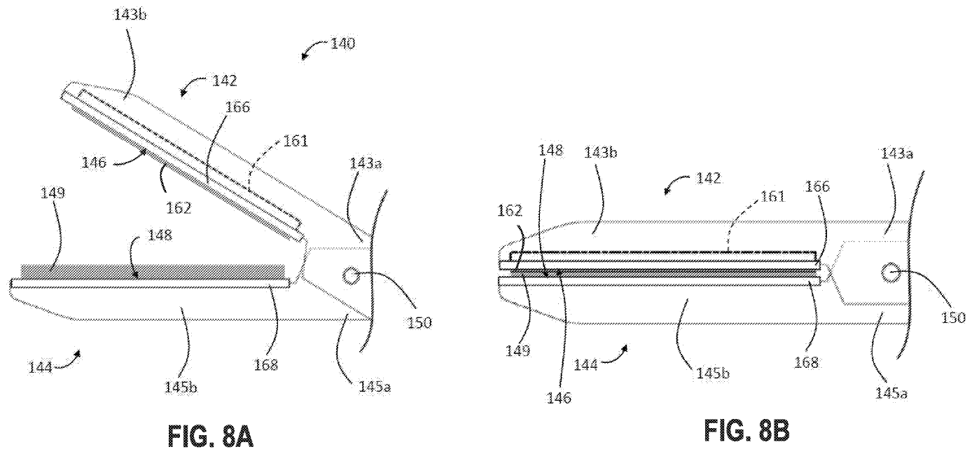

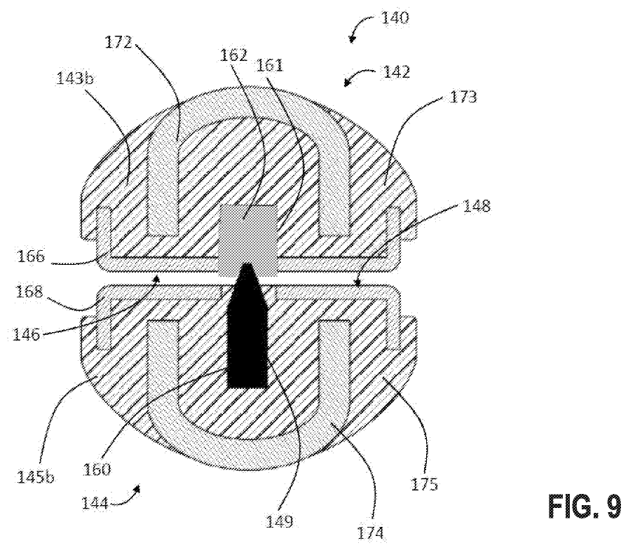

In configurations, jaw member 144 supports a longitudinally extending cutting electrode 149 in a slot 160 defined through tissue contacting surface 148 and a portion of distal body 145b of jaw member 144, while jaw member 142 includes a compression pad 162 (FIGS. 8A-9) disposed in a slot 161 (FIG. 9) defined through tissue contacting surface 146 and a portion of distal body 143b of jaw member 142. In such aspects, in the approximated position of jaw members 142, 144, cutting electrode 149 is urged into contact with compression pad 162 (FIGS. 8A-9) to grasp (and, in aspects, tension) tissue therebetween. Cutting electrode 149 may then be energized to cut the tissue disposed between cutting electrode 149 and compression pad 162 (FIGS. 8A-9). Cutting electrode 149 may additionally or alternatively be used to cut tissue in an open jaw configuration, e.g., with jaw members 142, 144 disposed in the spaced apart position. Cutting electrode 149 may be configured to be energized with monopolar Radio Frequency (RF) energy from a surgical generator (not shown) to conduct RF energy to tissue to cut the tissue, wherein the RF energy is returned to the generator to complete the circuit via a remote return device such as a return pad (not shown) or a local return device such as another portion of end effector assembly 140 or a separate instrument (not shown), e.g., a tenaculum, a probe, etc. Alternatively or additionally, cutting electrode 149 may be energized with bipolar RF energy wherein energy conducted from cutting electrode 149 to tissue is returned via either or both of tissue contacting surfaces 146, 148 of jaw members 142, 144, respectively, or other suitable local return device.

Referring still to FIG. 5, a drive rod 1484 is operably coupled to cam slot assembly 152 of end effector assembly 140, e.g., engaged with the cam pin thereof, such that longitudinal actuation of drive rod 1484 pivots jaw member 142 relative to jaw member 144 between the spaced apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced apart position. However, other suitable mechanisms and/or configurations for pivoting jaw member 142 relative to jaw member 144 between the spaced apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue contacting surfaces 146, 148 of jaw members 142, 144, respectively, are at least partially formed from an electrically conductive material and are energizable to different potentials to enable the conduction of RF electrical energy through tissue grasped therebetween, although tissue contacting surfaces 146, 148 may alternatively be configured to supply any suitable energy, e.g., thermal, microwave, light, ultrasonic, etc., through tissue grasped therebetween for energy based tissue treatment. Instrument 110 defines a conductive pathway (not shown) through housing 120 and shaft 130 to end effector assembly 140 that may include lead wires, contacts, and/or electrically conductive components to enable electrical connection of tissue contacting surfaces 146, 148 of jaw members 142, 144, respectively, and cutting electrode 149 (FIG. 9) to an energy source (not shown), e.g., an electrosurgical generator, for supplying energy to tissue contacting surfaces 146, 148 to treat, e.g., seal, tissue grasped between tissue contacting surfaces 146, 148 and to supply energy to cutting electrode 149 (FIG. 9) to treat, e.g., cut, tissue grasped between tissue contacting surfaces 146, 148 or otherwise positioned adjacent to cutting electrode 149 (FIG. 9).

With additional reference to FIGS. 6A-7, as noted above, actuation assembly 1100 is disposed within housing 120 and includes an articulation sub-assembly 1200, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Jaw drive sub-assembly 1400 is operably coupled between fourth input coupler 1140 of actuation assembly 1100 and drive rod 1484 such that, upon receipt of appropriate input into fourth input coupler 1140, jaw drive sub-assembly 1400 pivots jaw members 142, 144 between the spaced apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

Turning to FIGS. 8A-9, a distal portion of end effector assembly 140 is shown with jaw members 142, 144 disposed in the spaced apart position (FIG. 8A) and the approximated position (FIGS. 8B and 9). In aspects, either or both tissue contacting surfaces 146, 148 of jaw members 142, 144, respectively, are defined by respective tissue contacting plates 166, 168 disposed on the opposing surfaces of distal bodies 143b, 145b of jaw members 142, 144, respectively. As detailed above, jaw member 144 supports cutting electrode 149 in slot 160 defined through tissue contacting surface 148 (and through tissue contacting plate 168 and a portion of distal body 145b of jaw member 144), while jaw member 142 includes compression pad 162 disposed in slot 161 defined through tissue contacting surface 146 (and through tissue contacting plate 166 and a portion of distal body 143b of jaw member 142) and configured to oppose cutting electrode 149 in the approximated position of jaw members 142, 144 (FIGS. 8B and 9). Tissue contacting surfaces 146, 148 may define substantially U-shaped configurations wherein the slots 161, 160 defined therethrough terminate at positions proximally spaced from the distal ends of tissue contact surfaces 146, 148.

Cutting electrode 149 protrudes from jaw member 144 beyond tissue contacting plate 168 and towards jaw member 142. Compression pad 162 may protrude from tissue contacting surface 146 of tissue contacting plate 166 towards jaw member 144, may be recessed relative to tissue contacting surface 146 of tissue contacting plate 166, or may be substantially flush with tissue contacting surface 146 of tissue contacting plate 166. Compression pad 162 and jaw member 142 are configured, in conjunction with cutting electrode 149, such that, in the approximated position of jaw members 142, 144 (see FIGS. 8B and 9), cutting electrode 149 is urged into and at least partially compresses compression pad 162 (with tissue grasped therebetween), thus facilitating electrical tissue cutting upon activation of cutting electrode 149. The contact between cutting electrode 149 and compression pad 162 may also maintain a spacing between tissue contacting surfaces 146, 148 to inhibit electrical shorting via contact therebetween.

Either or both jaw members 142, 144 may include a structural jaw support 172, 174 defining the respective proximal flange 143a, 145a of the jaw member 142, 144 and extending into the respective distal body 143b, 145b. In such configurations, distal body 143b, 145b of either or both jaw members 142, 144 may further include jaw housings 173, 175 surrounding structural jaw supports 172, 174 and supporting tissue contacting plates 166, 168, respectively, thereon. Jaw housings 173, 175 may be formed from insulative materials and, in aspects, may be overmolded about jaw supports 172, 174 and a portion of tissue contacting plates 166, 168 to form jaw members 142, 144 and secure the components thereof to one another. In other configurations, jaw housings 173, 175 are conductive and electrically isolated from the other components of jaw members 142, 144 via suitable insulation. Alternatively or additionally, either or both jaw members 142, 144 may be formed from a monolithic, electrically conductive piece of material defining the structural jaw support, tissue contacting surface, and jaw housing thereof. At least a portion of the jaw housing, in such configurations, may be coated with an insulative material. Further, with respect to configurations where jaw member 144 is formed from a monolithic piece of material, cutting electrode 149 may be electrically isolated from the remainder of jaw member 144, e.g., via an insulator disposed therebetween. Thus, as utilized herein, reference to jaw housings 173, 175 includes insulative jaw housings, conductive jaw housings, and/or monolithic jaw structures defining jaw housings. Further, both jaw members 142, 144 may be similarly configured or may define different configurations, such as any combination of the jaw configurations detailed herein.

Continuing with reference to FIGS. 8A-9, as noted above, compression pad 162 and jaw member 142 are configured to facilitate electrical cutting of tissue grasped between jaw members 142, 144 upon activation of cutting electrode 149. More specifically, compression pad 162 is at least partially resiliently compressible and defines a suitable durometer, suitable durometer profile (e.g., with portions having different durometers), and/or suitable size and shape configuration to facilitate grasping tissue between compression pad 162 and cutting electrode 149 with sufficient force (and, in aspects, suitable tension) to enable effective and efficient electrical cutting of tissue upon activation of cutting electrode 149. Various configurations of compression pad 162 to facilitate effective and efficient electrical cutting of tissue upon activation of cutting electrode 149 are detailed below.

Compression pad 162 may define a substantially uniform shape and/or material(s) along the length of compression pad 162, across the width of compression pad 162, and/or through the depth of compression pad 162 such that compression pad 162 exhibits substantially similar properties, e.g., durometer, across these dimension(s). Alternatively, compression pad 162 may define a varied shape and/or be formed from different materials along the length of compression pad 162, across the width of compression pad 162, and/or through the depth of compression pad 162 such that compression pad 162 defines a particular durometer profile across these dimension(s).

Compression pad 162 may be formed from any suitable resiliently compressible material such as, for example, silicone or polytetrafluoroethylene (PTFE). Other suitable resiliently compressible materials having sufficient thermal properties are also contemplated for forming at least a portion of compression pad 162 such as, for example, resiliently compressible materials capable of withstanding temperatures of, in aspects, at least 200° C.; in other aspects, of at least 240° C.; or, in still other aspects, of at least 260° C. In aspects, compression pad 162 is formed from an overmold or injection moldable material or materials.

Compression pad 162, in aspects, may be formed from a single material or a substantially homogeneous mixture of materials. Alternatively or additionally, compression pad 162 may include filler materials disposed thereon (e.g., on the tissue contacting surface thereof) or therein (e.g., uniformly or non-uniformly distributed throughout compression pad 162). Such filler materials include, without limitation: calcium carbonate, talc, silica, wollastonite, clay, calcium sulfate fibers, mica, glass beads, and alumina trihydrate. Filler materials such as those noted above provide texture and/or roughness which increases gripping and reduces slippage of tissue grasped between compression pad 162 and cutting electrode 149 (FIG. 9). Such filler materials may also increase the effective durometer of compression pad 162, at least in the portions of compression pad 162 where such filler materials are provided. Other suitable filler materials or structures (including rods, columns, scaffolds, matrices, etc.) incorporated into compression pad 162 may provide increased structural support uniformly or selectively across one or more dimensions of compression pad 162 to achieve a particular effective durometer or effective durometer profile of compression pad 162. Likewise, the selective removal of material from on or within compression pad 162 and/or the formation of compression pad 162 with voids, channels, cut-outs, etc. uniformly or selectively across one or more dimensions of compression pad 162 may also be utilized to achieve a particular effective durometer or effective durometer profile of compression pad 162. Effective durometer, as utilized herein, refers to the ability of compression pad 162 to deform, e.g., to produce force at the engagement between compression pad 162 and cutting electrode 149.

In aspects, filler material may be provided for additional or alternative purposes. For example, graphite, metal fibers or powders, or other suitable filler materials may be provided to enhance the thermal conductivity of compression pad 162, e.g., to facilitate distribution of heat through jaw members 142, 144 to provide a more uniform tissue effect.

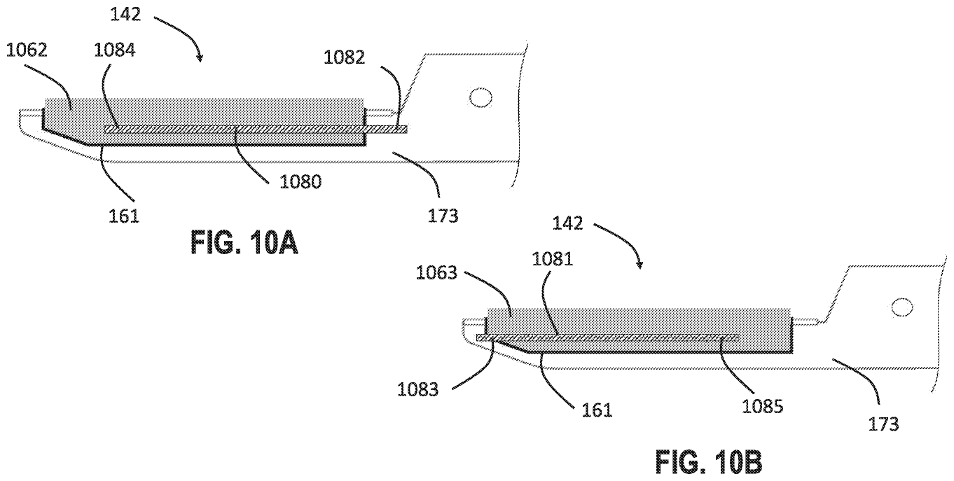

In addition to the configuration of compression pad 162 itself, the operable engagement of compression pad 162 within jaw member 142 (and the features that provide for this operable engagement) may also be configured to facilitate the grasping tissue between compression pad 162 and cutting electrode 149 with sufficient force (and, in aspects, suitable tension) to enable effective and efficient electrical cutting of tissue upon activation of cutting electrode 149. Various aspects and features of compression pads and/or the operable engagement of such compression pads within jaw member 142 to enable this effective and efficient electrical cutting of tissue upon activation of cutting electrode 149 are detailed below with reference to FIGS. 10A-15. To the extent consistent, any or all of these aspects and features may be used in any suitable combination with any or all of the other aspects and features.

Referring to FIG. 10A, a compression pad 1062 is shown at least partially disposed within slot 161 of jaw member 142 and operably engaged therein via a cantilever spring 1080. Compression pad 1062 may be similar to and include any of the features of compression pad 162 (FIGS. 8A-9) detailed above, except as explicitly contradicted below. Compression pad 1062 may be retained within slot 161 of jaw member 142 via cantilever spring 1080, as detailed below, and/or may be retained within slot 161 via engagement of compression pad 1062 with jaw housing 173 such as, for example, via mechanical engagement, press fitting, adhesion, molding, and/or in any other suitable manner.

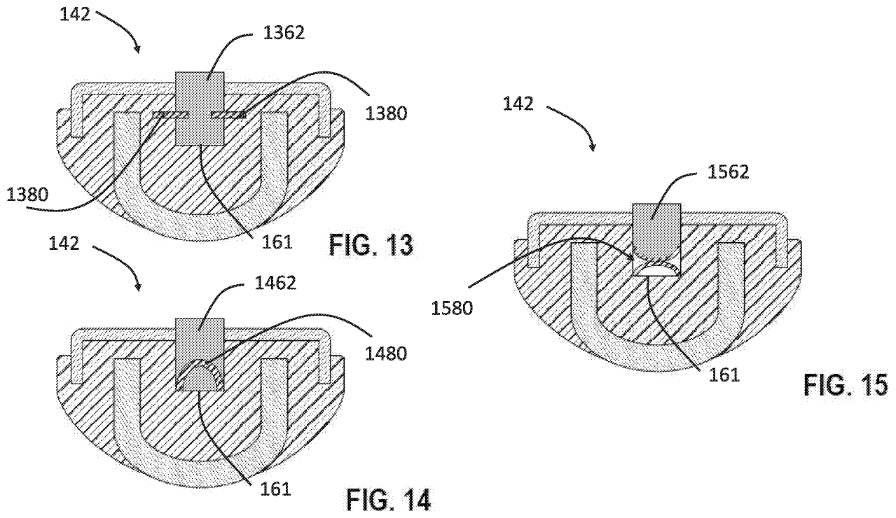

Cantilever spring 1080 includes a fixed end portion 1082 engaged within jaw member 142. Cantilever spring 1080 extends from fixed end portion 1082 into slot 161 of jaw member 142 to a free end portion 1084 of cantilever spring 1080. As shown in FIG. 10A, fixed end portion 1082 of cantilever spring 1080 is a proximal end portion of cantilever spring 1080 and cantilever spring 1080 extends distally, longitudinally through a portion of slot 161 of jaw member 142, to free end portion 1084 which is the distal end portion of cantilever spring 1080. However, other configurations are also contemplated such as, for example, those detailed below with reference to FIGS. 10B and 13. Further, although only a single cantilever spring 1080 is shown, it is contemplated that multiple cantilever springs 1080 be provided in the same configuration and/or different configurations. In aspects where multiple cantilever springs 1080 are provided, the cantilever springs 1080 may be transversely offset (along a width dimension of jaw member 142), vertically offset (along a height dimension of jaw member 142), oriented in different directions, and/or combinations thereof.

Cantilever spring 1080 may be formed from any suitable resiliently flexible material capable of enabling resilient flexion of free end portion 1084 of cantilever spring 1080 within slot 161 of jaw member 142 and relative to fixed end portion 1082 of cantilever spring 1080. For example, cantilever spring 1080 may be formed from steel, other suitable metal (including metal alloys), or a resiliently flexible polymeric material. Further, cantilever spring 1080 may define a cylindrical cross-sectional configuration, a flat or plate-like cross-sectional configuration, or any other suitable configuration (including varying cross-sectional configurations along the length).

Continuing with reference to FIG. 10A, cantilever spring 1080 extends into compression pad 1062 within slot 161 of jaw member 142 such that free end portion 1084 of cantilever spring 1080 is disposed within compression pad 1062. Compression pad 1062 may be pre-formed with an aperture defined at least partially therethrough for receipt of free end portion 1084 of cantilever spring 1080 to thereby operably engage compression pad 1062 about cantilever spring 1080. Alternatively, compression pad 1062 may be molded into slot 161 and about cantilever spring 1080 to capture free end portion 1084 of cantilever spring 1080 within compression pad 1062 to thereby operably engage compression pad 1062 about cantilever spring 1080. In still another configuration, compression pad 1062 may be disposed atop, rather than extend into, cantilever spring 1080. In such configurations, compression pad 1062 may sit atop cantilever spring 1080 or may be secured thereto in any suitable manner such as, for example, via adhesion, mechanical engagement, etc. In any of the above aspects, cantilever spring 1080 may extend through (or along) at least about 50%, at least about 60%, at least about 70%, or at least about 80% of a length of compression pad 1062.

Cantilever spring 1080 provides additional support and spring properties to compression pad 1062 (relative to jaw member 142), thereby changing the effective durometer of compression pad 1062. Further, due to the cantilever configuration of cantilever spring 1080, whereby greater flexion of cantilever spring 1080 is achieved in response to a force applied towards free end portion 1084 as compared to the same force being applied towards fixed end portion 1082, the effective durometer of compression pad 1062 is varied along the length of compression pad 1062. In the configuration of cantilever spring 1080 of FIG. 10A, the effective durometer of compression pad 1062 may decrease in a proximal-to-distal direction along the length of compression pad 1062 as cantilever spring 1080 extends away from fixed end portion 1082 thereof. Further, the distal portion of compression pad 1062 that is distal of and, thus, does not receive cantilever spring 1080, may provide a different effective durometer compared to the portion of compression pad 1062 that receives cantilever spring 1080. Cantilever spring 1080 (and additionally or alternatively any of spring components 1081, 1186, 1281a, 1281b, 1380, 1480, and/or 1580) may include variable dimensions along the length such as, for example, a varying thickness to thereby vary the spring properties and effective durometer of compression pad 1062.

With reference to FIG. 10B, a compression pad 1063 is shown at least partially disposed within slot 161 of jaw member 142 and operably engaged therein via a cantilever spring 1081. Compression pad 1063 and cantilever spring 1081 are similar to compression pad 1062 and cantilever spring 1080 (see FIG. 10A) and may include any of the features thereof detailed above except that fixed end portion 1083 of cantilever spring 1081 is a distal end portion of cantilever spring 1081 fixed within jaw housing 173 and cantilever spring 1081 extends proximally from fixed end portion 1083 into slot 161 of jaw member 142 and into compression pad 1063 to free end portion 1085 which defines the proximal end portion of cantilever spring 1081.

Thus, in the configuration of cantilever spring 1081 of FIG. 10B, the effective durometer of compression pad 1063 may decrease in a distal-to-proximal direction along the length of compression pad 1063 as cantilever spring 1081 extends away from fixed end portion 1083 thereof. Further, the proximal end portion of compression pad 1063 that is proximal of and, thus, does not receive cantilever spring 1081, may provide a different effective durometer as compared to the portion of compression pad 1063 that receives cantilever spring 1081.

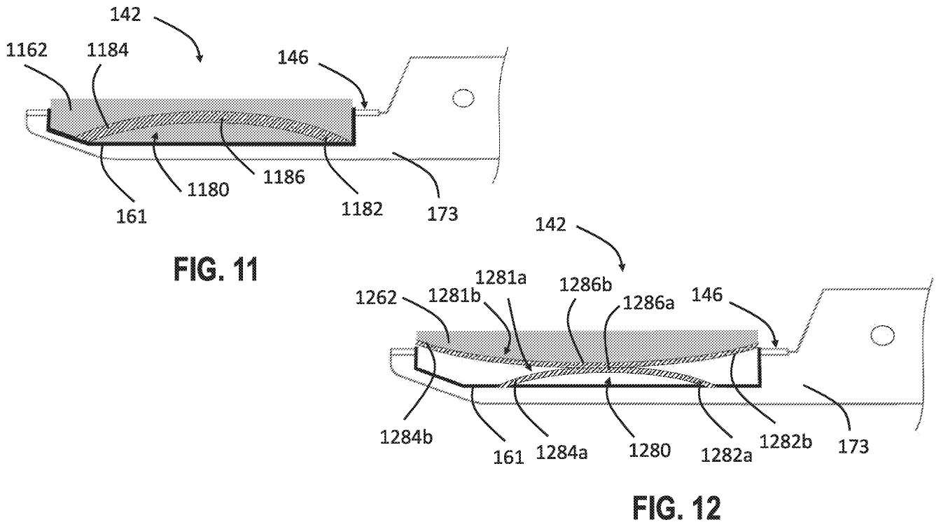

Turning to FIG. 11, a compression pad 1162 is shown at least partially disposed within slot 161 of jaw member 142 and operably engaged therein via an arched spring 1180. Compression pad 1162 may be similar to and include any of the features of compression pad 162 (FIGS. 8A-9) or compression pad 1062 (FIG. 10A), as detailed above, except as explicitly contradicted below. Compression pad 1162 may be retained within slot 161 of jaw member 142 via arched spring 1180, as detailed below, and/or may be retained within slot 161 via engagement of compression pad 1162 with jaw housing 173 such as, for example, via mechanical engagement, press fitting, adhesion, molding, and/or in any other suitable manner.

Arched spring 1180 is disposed within slot 161 of jaw member 142 and includes first and second end portions 1182, 1184 which, in the orientation of arched spring 1180 in FIG. 11, are respective proximal and distal ends of arched spring 1180. However, other configurations are also contemplated such as, for example, the configuration detailed below with reference to FIG. 14. End portions 1182, 1184 of arched spring 1180 may be fixed to jaw housing 173 or may be movable relative thereto. Arched spring 1180 includes an arch-shaped body 1186 extending between first and second end portions 1182, 1184 and defining a convex configuration oriented towards tissue contacting surface 146 of jaw member 142. Arched spring 1180 may be disposed at rest in the absence of force applied to compression pad 1162 inwardly into jaw member 142 (e.g., from tissue and/or cutting electrode 149 (FIG. 9)) or may be pre-loaded such that arched spring 1180 is maintained in a partially loaded condition in the absence of force applied to compression pad 1162 inwardly into jaw member 142.

Although only a single arched spring 1180 is shown, it is contemplated that multiple arched springs 1180 be provided in the same configuration and/or different configurations. In aspects where multiple arched springs 1180 are provided, the arched springs 1180 may be transversely offset (along a width dimension of jaw member 142), vertically offset (along a height dimension of jaw member 142 and/or defining different radii of curvature), oriented in different directions, and/or combinations thereof.

Arched spring 1180 may be formed from any suitable resiliently flexible material capable of enabling resilient flexion of body 1186 of arched spring 1180 within slot 161 of jaw member 142 and relative to end portions 1182, 1184 of arched spring 1180. For example, arched spring 1180 may be formed from steel, other suitable metal (including metal alloys), or a resiliently flexible polymeric material. Further, arched spring 1180 may define a cylindrical cross-sectional configuration, a flat or plate-like cross-sectional configuration, or any other suitable configuration (including varied cross-sectional configurations along the length).

Continuing with reference to FIG. 11, body 1186 of arched spring 1180 extends through compression pad 1162 within slot 161 of jaw member 142. Compression pad 1162 may be pre-formed with an aperture defined at least partially therethrough for receipt of arched spring 1180 to thereby operably engage compression pad 1162 about arched spring 1180. Alternatively, compression pad 1162 may be molded into slot 161 and about arched spring 1180 to capture body 1186 of arched spring 1180 within compression pad 1162 to thereby operably engage compression pad 1162 about arched spring 1180. In still another configuration, compression pad 1162 may be disposed atop, rather than extend into, arched spring 1180. In such configurations, compression pad 1162 may sit atop arched spring 1180 or may be secured thereto in any suitable manner such as, for example, via adhesion, mechanical engagement, etc.

In any of the above aspects, arched spring 1180 may extend through (or along) at least about 50%, at least about 60%, at least about 70%, at least about 80%, or a substantial entirety of a length of compression pad 1162. Either or both end portions 1182, 1184 of arched spring 1180 may be disposed at the end portions of compression pad 1162 may be offset within compression pad 1162, or may be offset outside of compression pad 1162. Further, the apex of the arched body 1186 of arched spring 1180 may be centered along a length of arched spring 1180, may be more proximally disposed along a length of arched spring 1180 compared to a center of compression pad 1162, or may be more distally disposed along a length of arched spring 1180 compared to the center of compression pad 1162.

Arched spring 1180 alters the effective durometer of compression pad 1162. Further, due to the configuration of arched spring 1180 defining a convex body 1186 extending between first and second end portions 1182, 1184 (fixed end portions 1182, 1184, in aspects) along the length of compression pad 1162, the effective durometer provided to compression pad 1162 is varied along the length of compression pad 1162. Thus, arched spring 1180 may be configured and positioned relative to compression pad 1162 to achieve a desired effective durometer profile of compression pad 1162 along the length thereof.

Referring to FIG. 12, a compression pad 1262 is shown at least partially disposed within slot 161 of jaw member 142 and operably engaged therein via a spring assembly 1280 including first and second arched springs 1281a, 1281b. Compression pad 1262 may be similar to and include any of the features of any of the compression pads detailed above, except as explicitly contradicted below. Compression pad 1262 may be retained within slot 161 of jaw member 142 via spring assembly 1280, as detailed below, and/or may be retained within slot 161 via engagement of compression pad 1262 with jaw housing 173 such as, for example, via mechanical engagement, press fitting, adhesion, molding, and/or in any other suitable manner.

Spring assembly 1280, as noted above, includes first and second arched springs 1281a, 1281b. First arched spring 1281a is disposed towards a bottom of slot 161 of jaw member 142 and includes first and second end portions 1282a, 1284a which, in the orientation of first arched spring 1281a in FIG. 12, are respective proximal and distal ends of first arched spring 1281a. However, other configurations are also contemplated such as, for example, the configuration detailed below with reference to FIG. 15. End portions 1282a, 1284a of first arched spring 1281a may be fixed to jaw housing 173 or may be movable relative thereto. First arched spring 1281a includes an arc-shaped body 1286a extending between first and second end portions 1282a, 1284a and defining a convex configuration oriented towards tissue contacting surface 146 of jaw member 142. First arched spring 1281a may be disposed at rest in the absence of force applied to compression pad 1262 inwardly into jaw member 142 (e.g., from tissue and/or cutting electrode 149 (FIG. 9)) or may be pre-loaded such that first arched spring 1281a is maintained in a partially loaded condition in the absence of force applied to compression pad 1262 inwardly into jaw member 142.

Second arched spring 1281b is disposed within slot 161 of jaw member 142 atop first arched spring 1281a and extends along a bottom surface of compression pad 1262. Compression pad 1262 may be secured to second arched spring 1281b along the bottom surface of compression pad 1262 via mechanical engagement, adhesion, molding, and/or in any other suitable manner. In aspects, rather than second arched spring 1281b extending along the bottom surface of compression pad 1262, second arched spring 1281b may extend at least partially through compression pad 1262, e.g., similarly as detailed above with respect to arched spring 1180 and compression pad 1162 (FIG. 11).

Second arched spring 1281b includes first and second end portions 1282b, 1284b which, in the orientation of second arched spring 1281b in FIG. 12, are respective proximal and distal ends of second arched spring 1281b. However, other configurations are also contemplated such as, for example, the configuration detailed below with reference to FIG. 15. Second arched spring 1281b includes an arc-shaped body 1286b extending between first and second end portions 1282b, 1284b and defining a concave configuration oriented towards tissue contacting surface 146 of jaw member 142. Thus, second arched spring 1281b is oriented oppositely as compared to first arched spring 1281a with the apexes thereof abutting one another. The apexes of first and second arched springs 1281a, 1281b may be welded to one another, adhered to one another, or attached to one another in any other suitable manner.

First and second arched springs 1281a, 1281b may be formed from any suitable resiliently flexible material (similar or different from one another) capable of enabling resilient flexion thereof within slot 161 of jaw member 142, such as, for example, the materials noted above. Further, first and second arched springs 1281a, 1281b may define a cylindrical cross-sectional configuration, a flat or plate-like cross-sectional configuration, or any other suitable configuration (similar or different from one another) (including varied cross-sectional configurations along the length).

Spring assembly 1280 provides a resilient coupling of compression pad 1262 with jaw member 142, thereby altering the effective durometer of compression pad 1262. Further, the effective durometer of compression pad 1262 varies along the length of compression pad 1262 due to the configuration of spring assembly 1280. Thus, spring assembly 1280 may be configured and positioned relative to compression pad 1262 to achieve a desired effective durometer profile of compression pad 1262 along the length thereof.

FIGS. 13-15 illustrate compression pads 1362, 1462, 1562 at least partially disposed within slot 161 of jaw member 142 and operably engaged therein via springs 1380, spring 1480, and springs 1580, respectively. In other aspects, components 1380, 1480, and/or 1580 are substantially rigid components rather than springs. Compression pads 1362, 1462, 1562 and corresponding springs 1380, 1480, 1580 are similar to the configurations of FIGS. 10A, 11, and 12, respectively, as detailed above, except that the orientations of springs 1380, 1480, 1580 are rotated about 90 degrees (and that two cantilever springs 1380 are provided in FIG. 13, although it is also contemplated that only one cantilever spring 1380 is provided). Thus, springs 1380, 1480, 1580 may be configured and positioned relative to compression pads 1362, 1462, 1562 to achieve a desired effective durometer profile of compression pads 1362, 1462, 1562 across the widths thereof.

Aspects of this disclosure may be further described by reference to the following numbered paragraphs:
1. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, and wherein the first jaw member includes a jaw body defining a slot, a compression pad at least partially disposed within the slot, and a cantilever spring operably coupling the compression pad with the jaw body.
2. The surgical end effector assembly according to paragraph 1, wherein the cantilever spring extends longitudinally along the first jaw member.
3. The surgical end effector assembly according to paragraph 2, wherein the cantilever spring includes a fixed proximal end portion fixed to the jaw body and a free distal end portion that extends distally into the slot.
4. The surgical end effector assembly according to paragraph 2, wherein the cantilever spring includes a fixed distal end portion fixed to the jaw body and a free proximal end portion that extends proximally into the slot.
5. The surgical end effector assembly according to paragraph 1, wherein the cantilever spring extends transversely across the first jaw member.
6. The surgical end effector assembly according to any preceding paragraph, wherein cantilever spring extends partially into the compression pad and terminates at a free end portion within the compression pad.
7. The surgical end effector assembly according to any preceding paragraph, wherein the cantilever spring operably couples the compression pad with the jaw body to define a varied effective durometer of the compression pad in at least one dimension thereof.
8. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, and wherein the first jaw member includes a jaw body defining a slot, a compression pad at least partially disposed within the slot, and an arched spring operably coupling the compression pad with the jaw body.
9. The surgical end effector assembly according to paragraph 8, wherein the arched spring defines a convex configuration oriented towards the second jaw member.
10. The surgical end effector assembly according to paragraph 8 or 9, wherein the arched spring extends longitudinally along the first jaw member.
11. The surgical end effector assembly according to paragraph 8 or 9, wherein the arched spring extends transversely across the first jaw member.
12. The surgical end effector assembly according to any one of paragraphs 8-10, wherein the arched spring defines first and second end portions and an arched body portion disposed therebetween, the arched body portion extending through the compression pad.
13. The surgical end effector assembly according to paragraph 12, wherein the first and second end portions of the arched spring are fixed to the jaw body.
14. The surgical end effector assembly according to any one of paragraphs 8-13, wherein the arched spring operably couples the compression pad with the jaw body to define a varied effective durometer of the compression pad in at least one dimension thereof.
15. A surgical end effector assembly, comprising: first and second jaw members including respective first and second tissue contacting surfaces, at least one of the first or second jaw members movable relative to the other of the first or second jaw members between a spaced apart position and an approximated position, wherein the second jaw member includes a cutting electrode extending from the second jaw member towards the first jaw member, and wherein the first jaw member includes a jaw body defining a slot, a compression pad at least partially disposed within the slot, and first and second springs, the first spring coupled to the jaw body, the second spring coupled to the compression pad, the first and second springs operably coupling the compression pad with the jaw body.
16. The surgical end effector assembly according to paragraph 15, wherein the first and second springs are oppositely oriented relative to one another.
17. The surgical end effector assembly according to paragraph 16, wherein the first and second springs are arched springs coupled to one another at apexes thereof.
18. The surgical end effector assembly according to any one of paragraphs 15-17, wherein the first and second springs extend longitudinally along the first jaw member.
19. The surgical end effector assembly according to any one of paragraphs 15-17, wherein the first and second springs extend transversely across the first jaw member.
20. The surgical end effector assembly according to any one of paragraphs 15-19, wherein the first and second springs operably couple the compression pad with the jaw body to define a varied effective durometer of the compression pad in at least one dimension thereof.

While several aspects of this disclosure have been shown in the drawings, it is not intended that this disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

1. A surgical end effector assembly (140), comprising:
first and second jaw members (142, 144) including respective first and second tissue contacting surfaces (146, 148), at least one of the first or second jaw members (142, 144) movable relative to the other of the first or second jaw members (142, 144) between a spaced apart position and an approximated position, wherein the second jaw member (144) includes a cutting electrode (149) extending from the second jaw member (144) towards the first jaw member (142), characterized in that:
the first jaw member (142) includes a jaw body (143b) defining a slot (161), a compression pad (162) at least partially disposed within the slot (161), and at least one spring (1080, 1081, 1180, 1281a, 1281b, 1380, 1480, 1580) operably coupling the compression pad (162) with the jaw body (143b).

2. The surgical end effector assembly (140) according to claim 1, wherein the at least one spring is a cantilever spring (1080, 1801) extending longitudinally along the first jaw member (142).

3. The surgical end effector assembly (140) according to claim 2, wherein the cantilever spring (1080) includes a fixed proximal end portion fixed to the jaw body (143b) and a free distal end portion that extends distally into the slot (161).

4. The surgical end effector assembly (140) according to claim 2, wherein the cantilever spring (1801) includes a fixed distal end portion fixed to the jaw body (143b) and a free proximal end portion that extends proximally into the slot (161).

5. The surgical end effector assembly (140) according to claim 1, wherein the at least one spring is a cantilever spring (1380) extending transversely across the first jaw member (142).

6. The surgical end effector assembly (140) according to claim 1, wherein the at least one spring is a cantilever spring (1080, 1081, 1380) extending partially into the compression pad (162) and terminating at a free end portion within the compression pad (162).

7. The surgical end effector assembly (140) according to claim 1, wherein the at least one spring (1080, 1081, 1180, 1281a, 1281b, 1380, 1480, 1580) operably couples the compression pad (162) with the jaw body (143b) to define a varied effective durometer of the compression pad (162) in at least one dimension thereof.

8. The surgical end effector assembly according to claim 1, wherein the at least one spring is an arched spring (1180, 1281a, 1480, 1580) defining a convex configuration oriented towards the second jaw member (144).

9. The surgical end effector assembly according to claim 1, wherein the at least one spring is an arched spring (1180, 1281a, 1281b) extending longitudinally along the first jaw member (142).

10. The surgical end effector assembly according to claim 1, wherein the at least one spring is an arched spring (1480, 1580) extending transversely across the first jaw member (142).

11. The surgical end effector assembly according to claim 1, wherein the at least one spring is an arched spring (1180, 1480) defining first and second end portions and an arched body portion disposed therebetween, the arched body portion extending through the compression pad (162).

12. The surgical end effector assembly according to claim 1, wherein the at least one spring includes first and second springs (1281a, 1281b, 1380, 1580) oppositely oriented relative to one another.

13. The surgical end effector assembly according to claim 12, wherein the first and second springs are arched springs (1281a, 1281b, 1580) coupled to one another at apexes thereof.

14. The surgical end effector assembly according to claim 1, wherein the at least one spring includes first and second springs (1281a, 1281b, 1380) that extend longitudinally along the first jaw member (142).

15. The surgical end effector assembly according to claim 1, wherein the at least one spring includes first and second springs (1580) that extend transversely across the first jaw member (142).
